(19) United States
(12) Patent Application Publication
(10) Pub. No.: US 2009/0163855 A1
(43) Pub. Date: Jun. 25, 2009

(54) INFUSION SYSTEM WITH ADAPTIVE USER INTERFACE

(75) Inventors: John Shin, La Canada, CA (US); Stuart L. Sundem, Valencia, CA (US); Linda I. Torres, Moorpark, CA (US)

Correspondence Address: LEE, HONG, DEGERMAN, KANG & WAIMEY, 660 S. FIGUEROA STREET, Suite 2300, LOS ANGELES, CA, 90017, US

(73) Assignee: MEDTRONIC MINIMED, INC.

(21) Appl. No.: 11/963,943

(22) Filed: Dec. 24, 2007

(51) Int. Cl. A61M 31/00

(52) U.S. Cl. 604/66

(57) ABSTRACT

An infusion system with an adaptive user interface is disclosed. The infusion system is interactive with the user and adaptive to the user's needs, incrementally guiding the user to enter information or perform a desired or necessary action in the infusion system. As a result, the user is not required to remember how to navigate through a series of menu structures to enter the information or perform the action.

### FIELD

This invention relates to infusion systems and more specifically to an infusion system having an adaptive user interface.

### BACKGROUND

Diabetes mellitus is the most common of endocrine disorders, and is characterized by inadequate insulin action. Diabetes mellitus has two principal variants, known as Type 1 diabetes and Type 2 diabetes. The latter is also referred to as DM/II (diabetes mellitus type 2). adult-onset diabetes, maturity-onset diabetes, or NIDDM (non-insulin dependent diabetes mellitus).

Over the years, body characteristics have been determined by obtaining a sample of bodily fluid. For example, diabetics often test for blood glucose levels. Traditional blood glucose determinations have utilized a finger stick method using a lancet to withdraw a small blood sample that is applied to a test strip for analysis by a blood glucose meter. These systems are designed to provide data at discrete points but do not provide continuous data to show variations in the characteristic between testing times. These discrete measurements are capable of informing a patient of his blood glucose values at a point in time. Thus, the patient has enough information to administer “correction” amounts of insulin to reduce his current blood glucose reading. However, these discrete readings are not able to provide enough information for any type of automatic or semi-automatic system of administering insulin based on blood glucose values.

Recently, a variety of implantable electrochemical sensors have been developed for detecting and/or quantifying specific agents or compositions in a patient's blood or interstitial fluid. For instance, glucose sensors are being developed for use in obtaining an indication of blood glucose levels in a diabetic patient. These glucose sensors connected (wired or wirelessly) to a blood glucose monitor can provide continuous glucose readings over a period of time, such as 3 to 6 days. Such readings are useful in monitoring and/or adjusting a treatment regimen which typically includes the regular administration of insulin to the patient.

Thus, blood glucose readings improve medical therapies with programmable medication infusion pumps of the external type, as generally described in U.S. Pat. Nos. 4,562,751; 4,678,408; and 4,685,903; or implantable medication infusion pumps, as generally described in U.S. Pat. No. 4,573,994, which are herein incorporated by reference. Typical thin film sensors are described in commonly assigned U.S. Pat. Nos. 5,390,671; 5,391,250; 5,482,473; and 5,586,553 which are incorporated by reference herein. See also U.S. Pat. No. 5,299,571. In addition, characteristic glucose monitors used to provide continuous glucose data are described in commonly assigned U.S. patent application Ser. No. 11/322,568 entitled “Telemetered Characteristic Monitor System and Method of Using the Same” filed on Dec. 30, 2005, which is herein incorporated by reference in its entirety. In addition, infusion pumps receiving sensor data are described in commonly assigned U.S. patent application Ser. No. 10/867,529 entitled “System for Providing Blood Glucose Measurements to an Infusion Device” filed on Oct. 14, 2004, which is herein incorporated by reference in its entirety.

As sensor technology improves, there is greater desire to use the sensor values to control the infusion of drugs and medicine, such as insulin in a closed loop or semi-closed loop system. Specifically, a closed loop system for diabetes entails a glucose sensor and an insulin infusion pump attached to a patient, wherein the delivery of insulin is automatically administered by a controller of the infusion pump based on the sensor's glucose value readings. A semi-closed loop system typically includes a patient intervention step, wherein the amount of insulin to be infused as calculated by the controller of the infusion pump requires patient modification and/or acceptance before delivery.

A current infusion system provides the patient with a user interface to control and monitor the delivery of insulin. Generally, the user interface includes a display screen and an input device for displaying and entering user commands and/or information. Typically, the infusion system implements a menu method, wherein a menu structure is displayed on the screen allowing the patient to navigate through screen menus to set into motion an action the patient desires.

Generally, when a patient wishes the infusion system to perform a certain function, the patient must select a function category from a menu including a plurality of function categories the infusion system is capable of performing. Upon selecting the category, a more specific menu including a more specific plurality of function categories will be displayed, one of which the patient must again select. This process continues until the patient reaches a desired menu containing the function category that will perform the patient's desired action when selected.

In one example, during infusion therapy management, a glucose sensor may be disconnected or lost, wherein the infusion system will notify the patient of the lost sensor via an alert or display message. Once notified, the patient may choose to identify and locate the sensor that is lost. In order to do so, the patient must find and select a SENSOR category from a MAIN menu including a plurality of menu items representing different function categories the infusion system is capable of performing. Upon selecting SENSOR, a SENSOR menu including a plurality of menu items related to sensor management will be displayed, one of which the patient must recognize and select. Accordingly, the patient must continue to navigate through a number of menus and menu items until the patient reaches a desired menu containing the category that will perform the patient's desired action when selected, i.e., locate the lost sensor.

Clearly, the menu structure of the current infusion system is problematic. First, the menu structure requires the patient to have extensive knowledge and command of the menu categories to enter any particular therapy-related information or perform an action required by the infusion system because of the complexity of the menu structure. Moreover, the menu structure does not provide any guidance to help the patient enter information or perform the required action. Therefore, what is needed is an infusion system that is interactive with the patient and is adaptive to the patient's needs, wherein the patient is incrementally guided by the system to help the patient enter information or perform any desired or necessary action in the infusion system. By simplifying menu navigation, the burden on the user to remember how to proceed to the next appropriate step is reduced.

### SUMMARY

In accordance with an embodiment of the present invention, an infusion system having a smart and adaptive user interface is described. Embodiments of the present invention include in an infusion system including an external infusion device, a sensor, and a transmitter for transmitting a sensor signal from the sensor to the external infusion device, a method for guiding action by a user when the external infusion device fails to receive the sensor signal from the sensor, the method comprising displaying an alarm indicating that the infusion device has failed to receive the sensor signal from the sensor for a period of time, determining a cause of the failure to receive the sensor signal from the sensor, and directing the user to perform at least one corrective action associated with the failure to receive the sensor signal from the sensor by providing the user with one or more outputs to which the user is to respond with a respective user input or action, wherein each successive one of the outputs is responsive to a previous user input or action.

Preferably, the alarm indicates that the infusion device has failed to receive the sensor signal from the sensor for a predetermined period of time. Alternatively, the alarm indicates that the infusion device has failed to receive the sensor signal from the sensor for a period of time set by the user.

In accordance with one embodiment of the present invention, wherein the cause of the failure to receive the sensor signal from the sensor relates to the sensor being located too far away from the infusion device, and the directing the user to perform at least one corrective action comprises directing the user to move the infusion device closer to the sensor, the method further comprising determining whether the infusion device is receiving the sensor signal from the sensor, and informing the user that the failure to receive the sensor signal from the sensor has been corrected if the infusion device is determined to be receiving the sensor signal from the sensor.

Preferably, if the infusion device is determined not to be receiving the sensor signal from the sensor, the directing the user to perform at least one corrective action further comprises directing the user to check a connection between the sensor and the transmitter. In one embodiment, the directing the user to perform at least one corrective action further comprises directing the user to replace the sensor if the failure to receive the sensor signal from the sensor has not been corrected after directing the user to check the connection between the sensor and the transmitter.

In accordance with another embodiment of the present invention, in an infusion system including an external infusion device and a sensor, a method for guiding action by a user when the sensor requires the user to perform a calibration, the method comprising displaying a notification when the sensor requires a calibration to be performed by the user, and directing the user to perform the calibration by providing the user with one or more outputs to which the user is to respond with a respective user input or action, wherein each successive one of the outputs is responsive to a previous user input or action. Preferably, the directing the user to perform the calibration further comprises instructing the user to obtain a calibration reference value for the sensor and guiding the user to enter the calibration reference value into the infusion device to calibrate the sensor.

In accordance with another embodiment of the present invention, in an infusion system including an external infusion device for delivering medication to a user and a sensor for measuring glucose levels in the user, a method for guiding action by the user when a glucose level is above a maximum or below a minimum boundary, the method comprising displaying an alarm when a glucose level of the user is above a maximum or below a minimum boundary, and directing the user to perform at least one corrective action associated with the alarm by providing the user with one or more outputs to which the user is to respond with a respective user input or action, wherein each successive one of the outputs is responsive to a previous user input or action.

Preferably, the directing the user to perform at least one corrective action further comprises asking the user to adjust settings on the infusion device related to therapy management of the user, and directing the user to adjust one or more of the settings on the infusion device related to the therapy management of the user. Preferably, the settings on the infusion device related to the therapy management of the user comprise at least one of bolus-related settings and basal-related settings. Preferably, the directing the user to adjust one or more of the settings on the infusion device related to the therapy management of the user comprises at least one of programming a temporary basal rate, modifying a basal rate pattern, programming a meal bolus, and programming a correction bolus.

In one aspect of the invention, the method further comprises suspending delivery of the medication to the user when the glucose level of the user is below the minimum boundary, and resuming delivery of the medication to the user after directing the user to adjust one or more of the settings on the infusion device related to the therapy management of the user. Preferably, the delivery of the medication to the user is suspended automatically when the glucose level of the user is below the minimum boundary. Alternatively, the delivery of the medication to the user is suspended based on input from the user.

In accordance with another embodiment of the present invention, in an infusion system including an external infusion device and a sensor, a method for guiding action by a user when a calibration reference value for the sensor entered by the user triggers a calibration error, the method comprising displaying an alarm when the calibration reference value for the sensor entered by the user triggers the calibration error, determining a cause of the calibration error, and directing the user to perform at least one corrective action associated with the calibration error by providing the user with one or more outputs to which the user is to respond with a respective user input or action, wherein each successive one of the outputs is responsive to a previous user input or action.

In one aspect of the invention, the determining the cause of the calibration error comprises asking whether glucose levels of the user are stable, and if the glucose levels of the user are not stable, the directing the user to perform at least one corrective action further comprises instructing the user to wait for a period of time, instructing the user to obtain a calibration reference value, and guiding the user to enter the calibration reference value into the infusion device to calibrate the sensor.

In another aspect of the invention, the determining the cause of the calibration error comprises asking whether the calibration reference value for the sensor was correctly entered by the user, and if the calibration reference value for the sensor was not correctly entered, the directing the user to perform at least one corrective action further comprises instructing the user to reenter the calibration reference value into the infusion device to calibrate the sensor.

In a further aspect of the invention, the determining the cause of the calibration error comprises asking whether the calibration reference value for the sensor was too old to calibrate the sensor, and if the calibration reference value for the sensor was too old to calibrate the sensor, the directing the user to perform at least one corrective action further comprises instructing the user to obtain a new calibration reference value for the sensor and guiding the user to enter the new calibration reference value into the infusion device to calibrate the sensor.

In yet another aspect of the invention, the determining the cause of the calibration error comprises asking whether sensor was recently inserted into the user, and if the sensor was recently inserted into the user, the directing the user to perform at least one corrective action further comprises instructing the user to wait for a period of time, instructing the user to obtain a new calibration reference value for the sensor, and guiding the user to enter the new calibration reference value into the infusion device to calibrate the sensor.

Other features and advantages of the invention will become apparent from the following detailed description, taken in conjunction with the accompanying drawings which illustrate, by way of example, various features of embodiments of the invention.

### DESCRIPTION OF THE DRAWINGS

A detailed description of embodiments of the invention will be made with reference to the accompanying drawings, wherein like numerals designate corresponding parts in the several figures.

FIG. 1 is a view of an infusion system located on a body in accordance with an embodiment of the present invention.

FIG. 2(a) is a perspective view of a glucose sensor system for use in an embodiment of the present invention.

FIG. 2(b) is a side cross-sectional view of the glucose sensor system of FIG. 2(a).

FIG. 2(c) is a perspective view of a sensor set of the glucose sensor system of FIG. 2(a) for use in an embodiment of the present invention.

FIG. 2(d) is a side cross-sectional view of the sensor set of FIG. 2(c).

FIG. 3 is a cross-sectional view of a sensing end of the sensor of FIG. 2(d).

FIG. 4 is a perspective view illustrating a preferred embodiment of a subcutaneous sensor insertion set and telemetered characteristic monitor transmitter device when mated together in relation to a characteristic monitor system.

FIG. 5 is a top view of the subcutaneous sensor insertion set and telemetered characteristic monitor transmitter device when separated.

FIG. 6 is a perspective view of an infusion device in accordance with an embodiment of the present invention.

FIG. 7 is a side view of an infusion set with the insertion needle pulled out, for use in an embodiment of the present invention.

FIG. 8 is an example of a basal rate profile broken up into three-hour intervals in accordance with an embodiment of the present invention.

FIG. 9 is a front view of an infusion device in accordance with an embodiment of the present invention.

FIG. 10 illustrates a method for guiding user action when a lost sensor error is detected in accordance with an embodiment of the present invention.

FIG. 11 illustrates a method for guiding user action when a weak sensor signal is detected in accordance with an embodiment of the present invention.

FIG. 12 illustrates a method for guiding user action when a sensor calibration is required in accordance with an embodiment of the present invention.

FIGS. 13A and 13B illustrate methods for guiding user action when a user glucose level above a maximum limit or below a minimum limit is detected in accordance with an embodiment of the present invention.

FIG. 14 illustrates a method for guiding user action when a sensor calibration error occurs in accordance with an embodiment of the present invention.

### DETAILED DESCRIPTION

As shown in the drawings for purposes of illustration, the invention is embodied in an infusion system for regulating the rate of fluid infusion into a body of a user based on an analyte concentration measurement taken from the body. The infusion system is interactive with the user and adaptive to the user's needs, incrementally guiding the user to enter information or perform a desired or necessary action in the infusion system. As a result, the user is not required to remember how to navigate through a series of menu structures to enter the information or perform the action. In particular embodiments, the invention is embodied in an infusion system for regulating the rate of insulin infusion into the body of a user based on a glucose concentration measurement taken from the body. However, it will be recognized that further embodiments of the invention may be used to infuse other medications or liquids into the body, such as chemicals, enzymes, antigens, hormones, vitamins, or the like, based on the levels of other body characteristics including analytes or agents, compounds or compositions, such as hormones, cholesterol, medication concentrations, viral loads, bacterial levels, or the like.

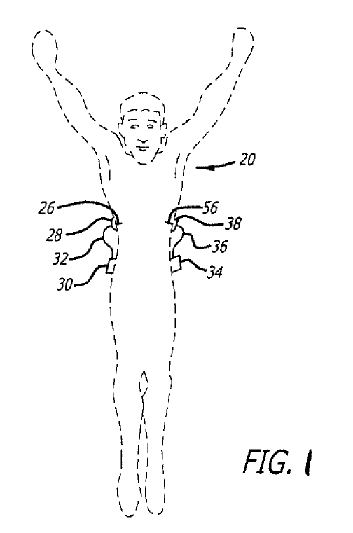

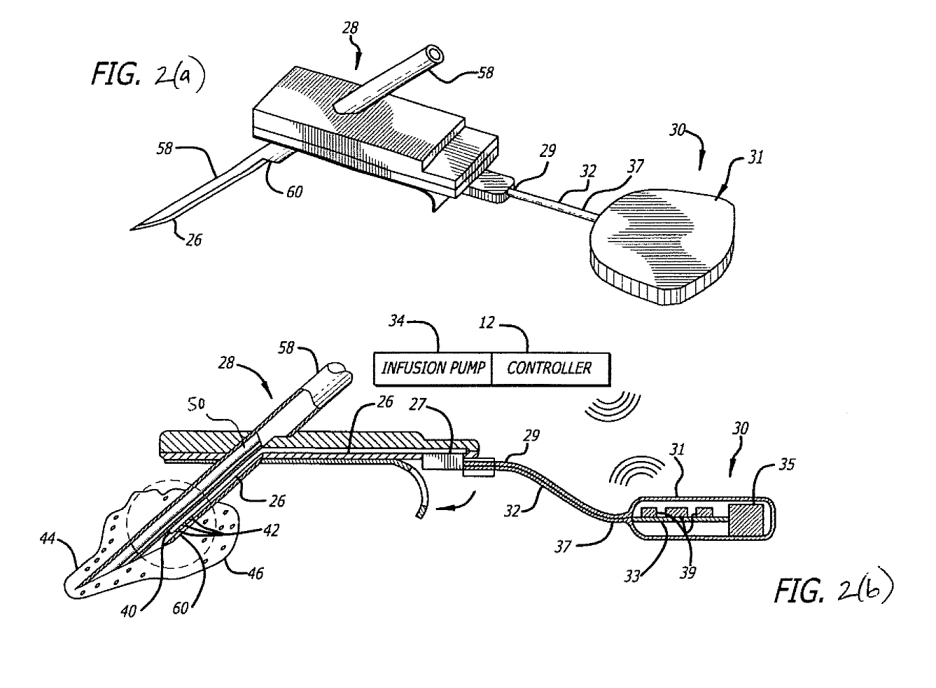

FIG. 1 is a view of an infusion system located on a body in accordance with an embodiment of the present invention. The infusion system includes a glucose monitoring system and a medication delivery system. The glucose monitoring system includes a sensor 26, a sensor set 28, a telemetered characteristic monitor transmitter 30, and a sensor cable 32, and the medication delivery system includes an infusion pump 34, an infusion tubing 36, and an infusion set 38, all worn on the body 20 of a user. The sensor 26 generates a sensor signal representative of blood glucose levels in the body 20, and provides the sensor signal to the telemetered characteristic monitor transmitter 30. The telemetered characteristic monitor transmitter 30 receives the sensor signal and transmits the sensor signal to a controller 12 (shown in FIG. 2b) of the infusion pump 34 via either a wireless (e.g., utilizing RF, IR, ultrasonic, or the like frequencies) or wired (not shown) connection.

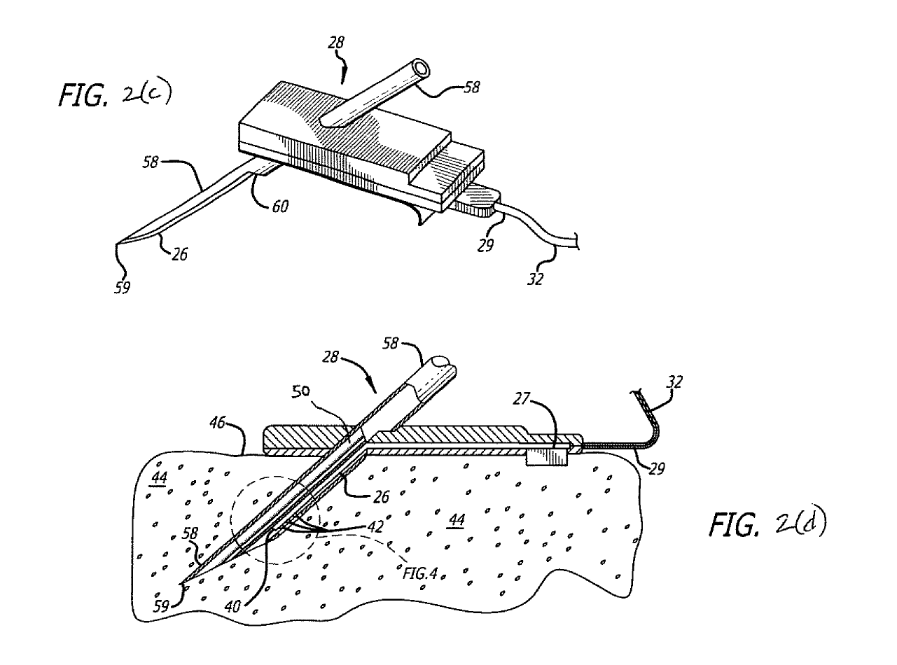

Referring to FIGS. 2(a) through 3, the sensor set 28 is provided for placement of an active portion of the sensor 26 at a selected site in the body of the user, preferably in the subcutaneous tissue of the user. However, in alternative embodiments, the sensor may be placed in other tissue types, such as muscle, lymph, organ tissue, veins, arteries, or the like. The sensor set 28 includes a hollow insertion needle 58 and a cannula 50. The needle 58 has a sharpened tip 59 to facilitate quick and easy subcutaneous placement of the cannula 50 at the insertion site.

Referring to FIGS. 2(a) and 2(b), the telemetered characteristic monitor transmitter 30 includes a transmitter housing 31 that supports a printed circuit board 33, batteries 35, antenna (not shown), and a sensor cable connector (not shown). Referring to FIGS. 2(b), 2(d) and 3, inside the cannula 50 is a sensing end 40 of the sensor 26 that has exposed electrodes 42 and is inserted through skin 46 into a subcutaneous tissue 44 of the user's body 20. The electrodes 42 are exposed through a window 60 formed in the cannula 50 to interstitial fluid (ISF) that is present throughout the subcutaneous tissue 44. Referring to FIGS. 2(b) and 2(d), the sensor 26 is held in place by the sensor set 28, which is adhesively secured to the user's skin 46. The sensor set 28 provides for a connector end 27 of the sensor 26 to connect to a first end 29 of the sensor cable 32. A second end 37 of the sensor cable 32 connects to the transmitter housing 31. The batteries 35 included in the transmitter housing 31 provide power for the sensor 26 and electrical components 39 on the printed circuit board 33. The electrical components 39 sample the sensor signal and store digital sensor values (Dsig) in a memory and then periodically transmit the digital sensor values Dsig from the memory to the controller 12, which is included in the infusion pump 34.

The sensor 26 may be an electro-enzymatic sensor, such as generally described in commonly assigned U.S. Pat. Nos. 5,390,671; 5,391,250; 5,482,473; and 5,586,553 which are incorporated by reference herein. See also U.S. Pat. No. 5,299,571. However, in alternative embodiments, the sensor may be other types of sensors, such as chemical based, optical based or the like. For example, other types of sensors are described in the following references: U.S. Provisional Application Ser. No. 60/007,515 to Van Antwerp et al. and entitled “Minimally Invasive Chemically Amplified Optical Glucose Sensor”; U.S. Pat. No. 6,011,984 issued Jan. 4, 2000 to Van Antwerp et al. and entitled “Detection of Biological Molecules Using Chemical Amplification”; and U.S. Pat. No. 6,766,183 issued Jul. 20, 2004 to Walsh et al. and entitled “Long Wave Flourophore Sensor Compounds and Other Fluorescent Sensor Compounds in Polymers”, all of which are herein incorporated by reference. Other compounds using Donor Acceptor fluorescent techniques may be used, such as disclosed in U.S. Pat. No. 5,628,310 issued May 13, 1997 to Rao et al. and entitled “Method and Apparatus to Perform Trans-cutaeous Analyte Monitoring”; U.S. Pat. No. 5,342,789 issued Aug. 30, 1994 to Chick et al. and entitled “Method and Device for Detecting and Quantifying Glucose in body Fluids”; and U.S. Pat. No. 5,246,867 issued Sep. 21, 1993 to Lakowicz et al. and entitled “Determination and Quantification of Saccharides by Luminescent Lifetimes and Energy Transfer”, all of which are herein incorporated by reference.

As shown in FIGS. 2(a)-2(d), the telemetered characteristic monitor transmitter 30 is coupled to a sensor set 28 by a sensor cable 32. In alternative embodiments, the cable 32 may be omitted, and the telemetered characteristic monitor transmitter 30 may include an appropriate connector for direct connection to the connector portion 27 of the sensor set 28 or the sensor set 28 may be modified to have the connector portion 27 positioned at a different location.

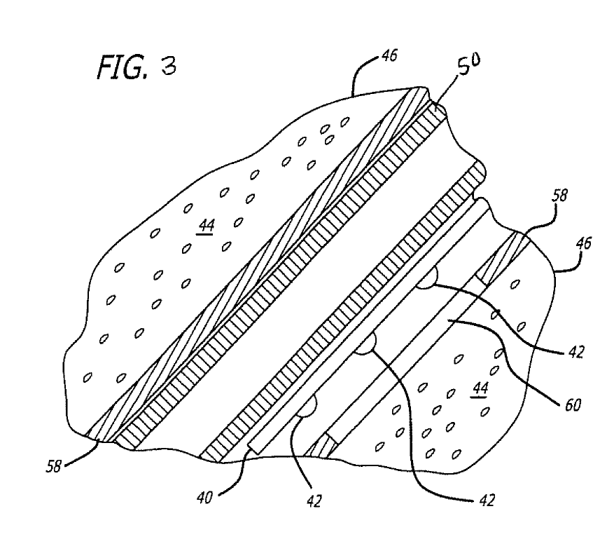

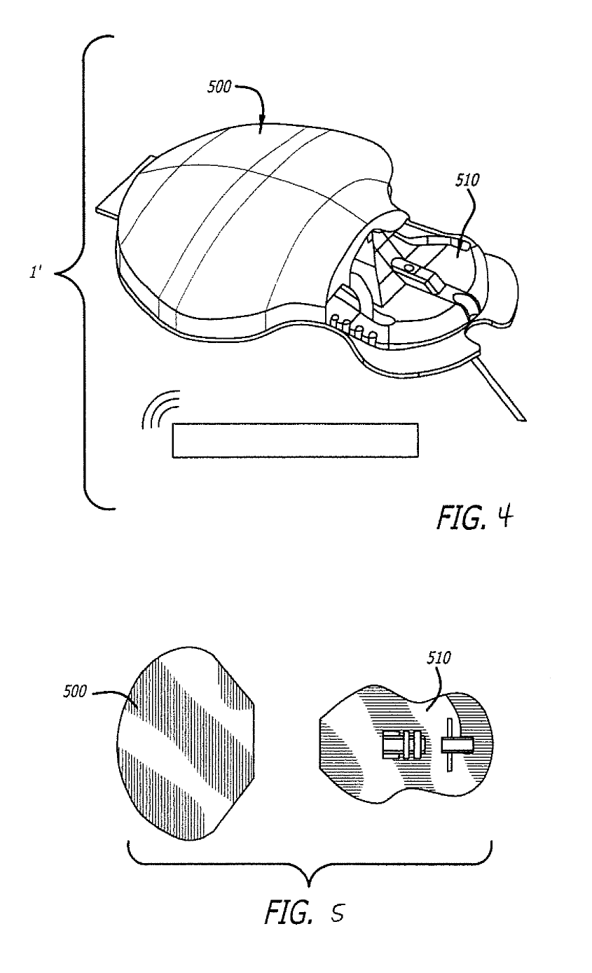

For example, FIGS. 4 and 5 show a possible alternative embodiment where characteristic monitor transmitter 500 and the sensor set 5 10 can be modified to allow a side-by-side direct connection between the characteristic monitor transmitter 500 and the sensor set 510 such that the characteristic monitor transmitter 500 is detachable from the sensor set 510, as shown in FIG. 5. Another possible embodiment (not shown) can modify the top of the sensor set 510 to facilitate placement of the telemetered characteristic monitor transmitter 500 over the sensor set 510.

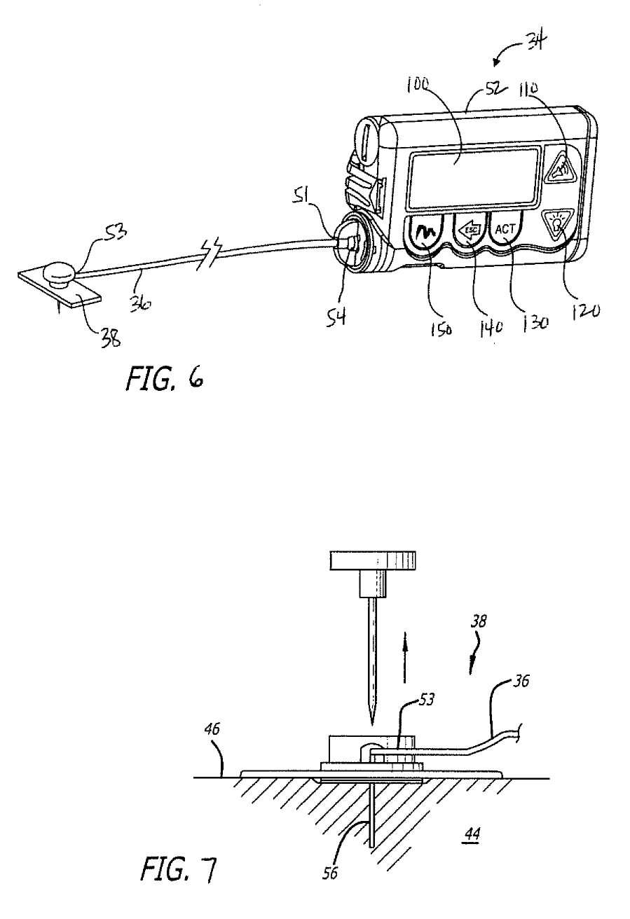

FIG. 6 is a perspective view of an infusion pump 34 for use in an embodiment of the present invention. The infusion pump 34 is preferably an external infusion pump worn on an exterior of the body of the user and is of the type generally described in U.S. Pat. Nos. 4,562,751; 4,678,408; 4,685,903; 5,080,653; 5,097,122; 5,505,709; 6,248,093; 6,362,591; 6,554,798; 6,555,986; and 6,752,787, which are incorporated by reference herein in their entirety. However, in alternative embodiments, the infusion pump may include separate durable and disposable housing portions that selectively engage and disengage from each other and may be of the type generally described in U.S. Provisional Application Ser. No. 60/678,290 filed May 6, 2005 and entitled “Infusion Device and Method with Disposable Portion,” U.S. application Ser. No. 11/211,095 filed Aug. 23, 2005 and entitled “Infusion Device and Method with Disposable Portion,” U.S. application Ser. No. 11/210,467 filed Aug. 23, 2005 and entitled “Infusion Device and Method with Drive Device in Infusion Device and Method with Drive Device in Separable Durable Housing,” U.S. Provisional Application Ser. No. 60/839,821 filed Aug. 23, 2006 and entitled “Systems and Methods Allowing for Reservoir Filling and Infusion Medium Delivery,” U.S. Provisional Application Ser. No. 60/839,822 filed Aug. 23, 2006 and entitled “Infusion Medium Delivery Device and Method with Drive Device for Driving Plunger in Reservoir,” U.S. Provisional Application Ser. No. 60/839,832 filed Aug. 23, 2006 and entitled “Infusion Medium Delivery Device and Method with Compressible or Curved Reservoir or Conduit,” U.S. Provisional Application Ser. No. 60/839,840 filed Aug. 23, 2006 and entitled “Infusion Medium Delivery System, Device and Method with Needle Inserter and Needle Inserter Device and Method,” and U.S. Provisional Application Ser. No. 60/839,741 filed Aug. 23, 2006 and entitled “Infusion Pumps and Methods and Delivery Devices and Methods with Same,” all of which are herein incorporated by reference. In further alternative embodiments, the infusion pump may be an implantable infusion pump, such as generally described in U.S. Pat. No. 4,573,994, which is herein incorporated by reference, or a system that uses a combination of implantable and external components.

Referring to FIGS. 1 and 6, the infusion pump 34 comprises a housing 52 that contains the controller 12 for processing digital sensor values Dsig received from the telemetered characteristic monitor transmitter 30 of the glucose monitoring system and generates commands for the infusion pump 34. Preferably, the controller 12 sends information to, or receives information from, a memory (not shown) housed in the housing 52. The infusion pump 34 further comprises a liquid crystal display (LCD) 100 for viewing pump information. The memory stores programs, historical data, user defined information, and control parameters. In preferred embodiments, the memory is a Flash memory and SRAM. However, in alternative embodiments, the memory may include other memory storage devices such as ROM, DRAM, RAM, EPROM, dynamic storage such as other flash memory, energy efficient hard drive, or the like.

In one embodiment, the infusion system is an open loop system. In such an open loop system, the controller 12 causes information about the user's blood glucose levels to be displayed on the LCD 100 of the infusion pump 34 based on the sensor signal received from the telemetered characteristic monitor transmitter 30 of the glucose monitoring system. At any desired time, the user or a caregiver (e.g., physician, parent, or the like) may view the user's blood glucose levels as measured by the glucose monitoring system on the LCD 100 of the infusion pump 34. In response to or independent of the displayed information, the user or caregiver may program the infusion pump 34 to infuse insulin into the body 20. For example, as described in commonly assigned U.S. Pat. No. 6,554,798 entitled “External Infusion Device with Remote Programming, Bolus Estimator and/or Vibration Alarm Capabilities.” which is incorporated by reference herein in its entirety, the controller 12 may calculate an estimated amount of insulin to be infused based upon the amount of carbohydrates to be ingested by the user, the user's carbohydrate ratio, the user's current blood glucose level as provided by a blood glucose meter, the user's target blood glucose level, the user's insulin sensitivity, and/or the amount of insulin on board (i.e., insulin already delivered to and still active in the user's body).

In other embodiments, the infusion system may be a closed loop or semi-closed loop system. In a closed loop system, after receiving the sensor signal from the telemetered characteristic monitor transmitter 30 of the glucose monitoring system, the controller 12 generates commands for infusing insulin into the body 20. The infusion pump 34 then infuses insulin into the body 20 accordingly. Alternatively, in a semi-closed loop system, the commands may be confirmed by the user before the infusion pump 34 infuses the insulin.

In additional embodiments, the infusion system may be used only for overnight closed-loop applications, where there is no expectation of any carbohydrates ingested. Instead, the focus may be to prevent hypoglycemic excursions during sleeping times because the immediate risks of hypoglycemia are much greater than hyperglycemia. Hypoglycemia can cause a person to pass out in 15 or 30 minutes while it takes hours for the severe effects of hyperglycemia to become evident and cause problems. In such an application, after receiving the sensor signal from the telemetered characteristic monitor transmitter 30 of the glucose monitoring system, the controller 12 generates commands for infusing insulin into the body 20. For example, the controller 12 may simply lower the basal rate or shut off the basal rate completely to prevent the blood glucose levels from falling to dangerous hypoglycemic levels. The controller 12 may also correct for hyperglycemic excursions by increasing the basal rate.

Yet in further embodiments, the infusion system may be a combination closed loop/open loop system. For example, the controller 12 may be programmed to function as an open loop system during; meal times (i.e., administration of meal boluses) or correction boluses, where the user or caregiver programs the infusion pump 34 to infuse a certain amount of insulin into the body. However, the controller 12 may return to a default closed-loop/semi-closed system when the insulin on board from a meal or correction bolus is de minimis (such as 4 to 6 hours), where the controller 12 generates commands for infusing insulin into the body based on the sensor signal received from the telemetered characteristic monitor transmitter 30 of the glucose monitoring system.

In accordance with the present invention, a user may navigate through a menu structure displayed on the LCD 100 by pressing a sequence of one or more keys (110, 120, 130, 140 and 150) to access and/or modify control parameters and data that have been stored in the memory. The control parameters and data may include basal parameters, bolus parameters, priming parameters, alarms, limits, infusion set feedback, personal identification information, historical data (such as the times and amounts of the latest dosages, program changes, when priming occurred, and the like), power supply status, reservoir status, and the like. The controller 12 uses the control parameters to calculate and issue the commands that affect the rate and/or frequency that a drive mechanism (not shown) forces fluid out of a reservoir, and into tubing 36 connected to an infusion set 38 that provides a fluid path into the user's body.

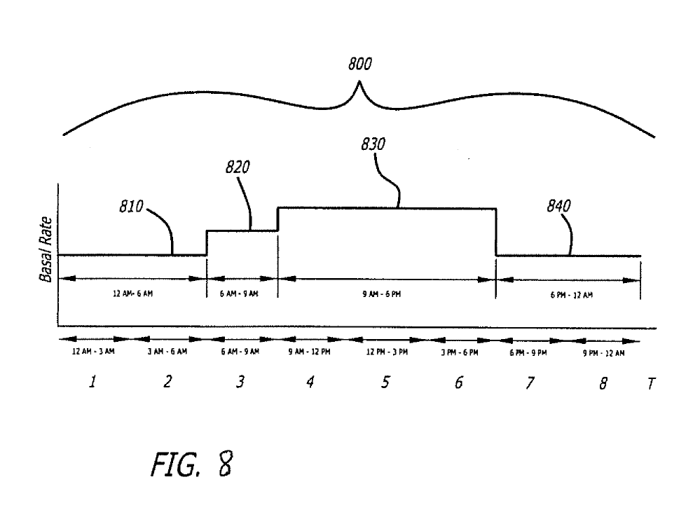

Preferably, the drive mechanism comprises a plunger slider that is adapted to couple with a piston, which is part of the reservoir located inside the infusion pump 34. The plunger slider moves the piston, which in turn forces fluid out of the reservoir. In particular embodiments, a connector tip 54 of the reservoir extends through the infusion device housing 52 and a first end 51 of the infusion tubing 36 is attached to the connector tip 54. A second end 53 of the infusion tubing 36 connects to the infusion set 38. Referring to FIGS. 1, 6 and 7, insulin is forced through the infusion tubing 36 into the infusion set 38 and into the body 20. The infusion set 38 is adhesively attached to the user's skin 46. As part of the infusion set 38, a cannula 56 extends through the skin 46 and terminates in the subcutaneous tissue 44, completing fluid communication between the reservoir and the subcutaneous tissue 44 of the user's body 20.

The LCD 100 displays menus, control parameters, options, operating modes, statuses, data, alarms, warnings, information, error messages, and the like. In preferred embodiments, the LCD 100 has sufficiently fine resolution to display words and numbers and to show graphics such as a meter bar or a sliding scale to indicate, for example, the amount of power remaining in the power supply, or the amount of medicament remaining in the reservoir, how far an individual has scrolled through a list of data, and the like.

Preferably, the LCD 100 has a backlight that the individual may activate to illuminate the LCD 100 as needed. In alternative embodiments, the LCD 100 may be replaced with an LED (light emitting diode) display, plasma screen, a touch screen, a color LCD, or the like. Also, the display resolution may be increased to display icons to represent data, control parameters, function keys, and the like. In other alternative embodiments, instead of or in addition to the LCD 100, feedback may be provided to the individual through sound, vibration, braille, or visually displayed on another device that has received information from the infusion pump 34. 10060] As shown in FIG. 6, the infusion pump 34 has five keys including an Up-Arrow key (scroll button) 110, a Down-Arrow key (scroll button) 120, an ACT key (activate button) 130, an Esc key (escape button) 140, and an Express Bolus key (shortcut button) 150. The keys provide the primary means for the individual to provide input to the infusion device 34. The individual presses the keys to display and scroll through information, call up menus, select menu items, select control parameters, change control parameters (change values or settings), enter information, turn on the backlight, and the like. In alternative embodiments, the infusion device 34 may utilize more or less keys or have different key arrangements than those illustrated. In other alternative embodiments, other types of input interfaces, such as buttons, a keyboard, mouse, joystick, voice activated controller, a touch screen, or the like, may be used. In further alternative embodiments, the keys 110, 120, 130, 140, and 150 may be omitted, and the LCD 100 may be used as a touch screen input device. Furthermore, devices other than the infusion pump 34, such as an RF programmer, a computer connected to a cradle, a PDA (Personal Digital Assistant), a phone, or the like may be used to provide an interface between the individual and the infusion pump 34.

A power supply of a preferred embodiment provides the power to operate the infusion pump 34, and in preferred embodiments, the power supply is at least one battery. In particular embodiments, the power supply is one or more replaceable AAA batteries. Energy storage devices such as capacitors, backup batteries, or the like provide temporary power to maintain the memory during power supply replacement. In alternative embodiments, the power supply is one or more button batteries, zinc air batteries, alkaline batteries, lithium batteries, lithium silver oxide batteries, AA batteries, or the like. In still further alternative embodiments, the power supply is rechargeable.

In alternative embodiments, the infusion system can be a part of a hospital-based glucose management system. Given that insulin therapy during intensive care has been shown to dramatically improve wound healing, reduce blood stream infections, renal failure, and polyneuropathy mortality, irrespective of whether subjects previously had diabetes, the present invention can be used in a hospital setting to control the blood glucose level of a patient in intensive care.

In these alternative embodiments, since an IV hookup is typically implanted into a patient's arm while the patient is in an intensive care setting (e.g., ICU), a glucose control system can be established which piggy-backs off the existing IV connection. Thus, in a hospital based system, intravenous (IV) catheters which are directly connected to a patient vascular system for purposes of quickly delivering IV fluids, can also be used to facilitate blood sampling and direct infusion of substances (e.g., insulin, anticoagulants) into the intra-vascular space. Moreover, glucose sensors may be inserted through the IV line to give real-time glucose levels from the blood stream.

Therefore, depending on the type of hospital based system, the alternative embodiments would not necessarily need the described system components, such as the sensor 26, the sensor set 28, the telemetered characteristic monitor transmitter 30, the sensor cable 32, the infusion tube 36, and the infusion set 38 as described in the preferred embodiments. Instead, standard blood glucose meters or vascular glucose sensors as described in patent application entitled “Multi-lumen Catheter,” filed Dec. 30, 2002, Ser. No. 10/331,949, which is incorporated by reference herein in its entirety, can be used to provide the blood glucose values to the infusion pump and the existing IV connection can be used to administer the insulin to the patient.

In preferred embodiments, the infusion pump 34 delivers steady amounts of insulin, known as a basal rate, throughout a day. The basal rate delivers the amount of insulin needed in a fasting state to maintain target glucose levels. The basal rate insulin is intended to account for the baseline insulin needs of the body, and makes up approximately fifty percent of the body's total daily insulin requirements.

The infusion pump 34 delivers basal rate insulin continuously over the twenty-four hours in the day. The infusion pump 34 can be set to automatically provide one or more different rates during different time intervals of the day. These different basal rates at various time intervals during the day usually depend on a patient's lifestyle and insulin requirements. For example, many insulin delivery system users require a lower basal rate overnight while sleeping and a higher basal rate during the day, or users may want to lower the basal rate during the time of the day when they regularly exercise.

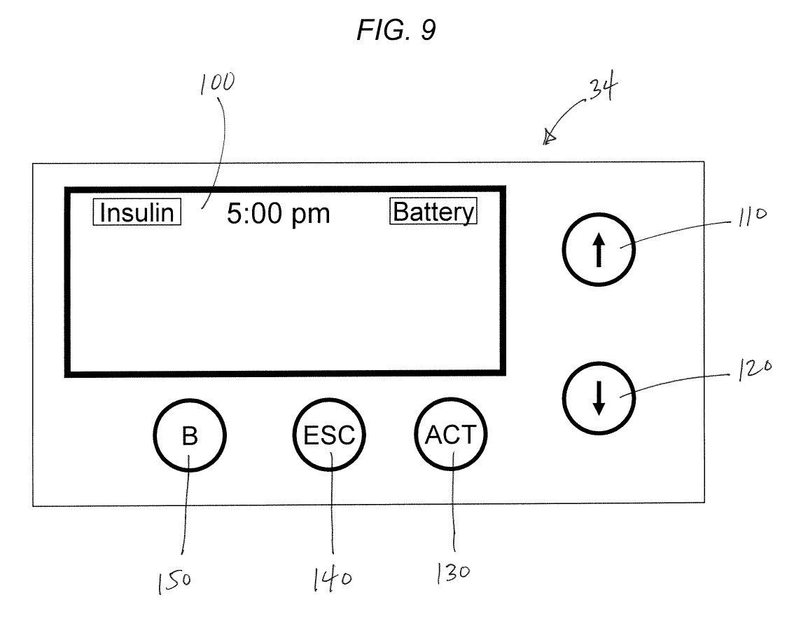

FIG. 8 is an example of a basal rate profile broken up into three-hour intervals in accordance with an embodiment of the present invention. Referring to FIG. 8, the basal pattern 800 can have various basal rates (810, 820, 830, 840) throughout the day, and the basal rates do not necessarily change at each interval. Moreover, adjustments to the specific basal rates can be made for each time interval. Notably, these intervals can be started at any time to match the user's schedule and intervals can be greater or less than three-hours in length. A single basal rate interval can be as short as a minimum basal rate interval capable of being programmed by an insulin delivery system, such as 30 minutes, or have a maximum of 24 hours.

A bolus is an extra amount of insulin taken to cover a rise in blood glucose, often related to a meal or snack. Whereas a basal rate profile provides continuously pumped small quantities of insulin over a long period of time, a bolus provides a relatively large amount of insulin over a fairly short period of time. Most boluses can be broadly put into two categories: meal boluses and correction boluses. A meal bolus is the insulin needed to control the expected rise in glucose levels due to a meal. A correction bolus is the insulin used to control unexpected highs in glucose levels. Often a correction bolus is given at the same time as a meal bolus because patients often notice unexpected highs in glucose levels when preparing to deliver a meal bolus related to meal.

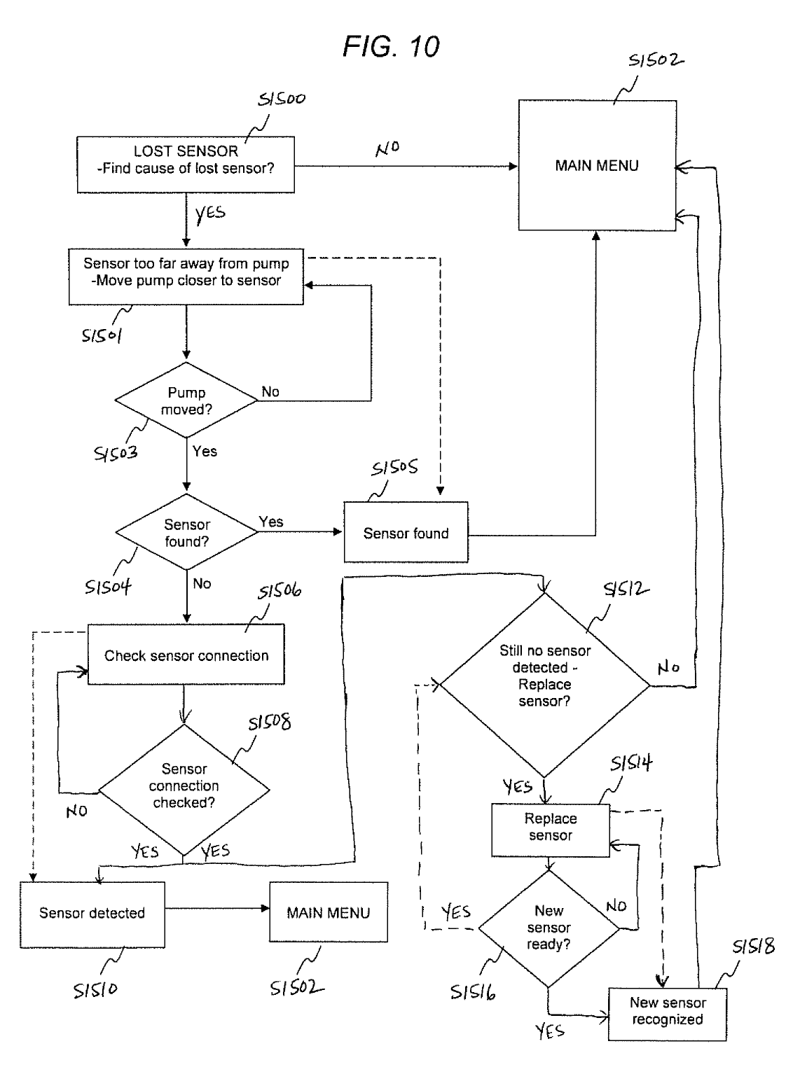

FIG. 9 illustrates a front view of an infusion device in accordance with one embodiment of the present invention. Here, the infusion device depicted is similar to the infusion device described with reference to FIG. 6. Referring to FIG. 9, the infusion device 34 comprises a display 100, scroll buttons 110, 120, an activate button 130, an escape button 140 and a shortcut button 150. The display 100 displays information related to infusion therapy management. Preferably, the information is arranged in a menu format, such that a user may select various menu items in order to enter therapy-related information or activate therapy-related functions and settings. In alternative embodiments, the infusion device 34 may be another device that is capable of communicating with and displaying information about the user's blood glucose levels based on the sensor signal received from the telemetered characteristic monitor transmitter 30 of the glucose monitoring system, such as a remote programmer for the infusion device 34, a dedicated display unit, or the like.

The scroll buttons 110, 120 are used to scroll up and down through a list of menu items displayed on the display 100. The scroll buttons 110, 120 may also be used to answer yes/no questions displayed on the display 100. For example, the upper scroll button 110 may be used to enter a “yes” answer and the lower scroll button 120 may be used to enter a “no” answer. The scroll buttons 110, 120 are also useful when entering numerical digits into the infusion device 34. For example, when prompted to enter the number “8”, the upper scroll button 110 may be continually pressed to incrementally change a number initially appearing as “0” on the display 100 to the number “8.”

When pressed, the activate button 130 accepts a selected menu item or activates a selected setting. The escape button 140 returns the display 100 to a previous screen when pressed. The escape button 140 may also cancel settings if the activate button 130 is not yet pressed. In an alternative embodiment, the activate button 130 and the escape button 140 may individually or collectively be used to answer yes/no questions displayed on the display 100. For example, the activate button 130 may be used to enter a “yes” answer and the escape button 140 may be used to enter a “no” answer. The shortcut button 150 may be used to directly access a desired menu without having to navigate through a plurality of hierarchical menus.

Embodiments of the present invention provide methods for guiding user entry of information or performance of a particular action on the infusion device. As a result, the user is not required to remember how to navigate through a series of menu structures to enter the information or perform the action.

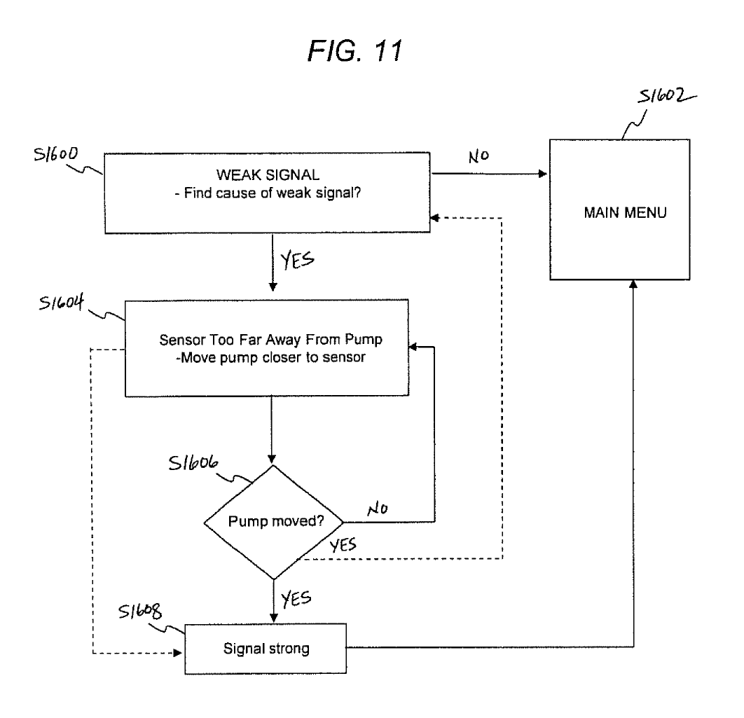

FIG. 10 illustrates a method for guiding user action when a sensor is lost in accordance with one embodiment of the present invention. As described above, the telemetered characteristic monitor transmitter 30 transmits a sensor signal to an infusion device 34, and information about the user's blood glucose levels may be displayed on the infusion device 34 based on the received sensor signal. However, if the infusion device 34 fails to receive the sensor signal from the transmitter 30 for a predetermined period of time (e.g., 40, 45, or 60 minutes), then the infusion device 34 may display a LOST SENSOR alarm screen to notify the user that the infusion device 34 is no longer communicating with the glucose monitoring system (S1500). Concurrently with or after the LOST SENSOR alarm screen is displayed, the user is asked whether the cause of the lost sensor is to be found (S1500). If the answer is “no,” a MAIN MENU screen is displayed (S1502). If the answer is “yes,” the infusion device 34 attempts to identify the cause of the lost sensor.

In accordance with one embodiment of the present invention, the infusion device 34 ascertains that the sensor is too far away from the infusion device 34. Accordingly, the user is informed of this information and is instructed to move the infusion device closer to the sensor (S1501). Thereafter, if the infusion device determines that it is again receiving the sensor signal from the sensor, the user is informed that the sensor is found (S1505), and the MAIN MENU screen is displayed (S1502).

Alternatively, the infusion device may ask the user if the infusion device has been moved (S1503). If the answer is “no,” the user is brought back to the screen instructing the infusion device to be moved (S1501). However, if the infusion device has been moved, the infusion device determines whether it is again receiving the sensor signal from the sensor, and if so, the user is informed that the sensor is found (S1505), and the MAIN MENU screen is displayed (S1502).

However, if the sensor is not found, the user is instructed to check the sensor connection (S1506). The user may then inspect the site at which the sensor is attached and repair any damaged or loose connection. Thereafter, if the user is successful in repairing the connection and the infusion device determines that it is again receiving the sensor signal from the sensor, the infusion device 34 may inform the user that the sensor is detected (S1510), and subsequently display the MAIN MENU screen (S1502).

Alternatively, after the user attempts to repair the sensor connection, the infusion device 34 may ask the user if the sensor connection has been checked (S1508). If the user answers “no,” the user is brought back to the screen instructing the user to check the sensor connection (S1506). If the user answers “yes,” the infusion device determines whether it is again receiving the sensor signal from the sensor, and if so, the infusion device 34 may inform the user that the sensor is detected (S1510), and subsequently display the MAIN MENU screen (S1502).

Otherwise, the user is informed that the sensor is still not detected, and then asked whether the sensor is to be replaced (S1512). If the answer is “no,” the MAIN MENU screen is displayed (S1502). If the answer is “yes,” the user is instructed to replace the sensor (S1514). If the sensor is successfully replaced and the infusion device determines that it is again receiving the sensor signal from the sensor, the infusion device 34 may inform the user that the new sensor is recognized (S1518), and then display the MAIN MENU screen (S1502).

Alternatively, the infusion device 34 may ask the user if the new sensor is ready (S1516). If the sensor is not yet replaced, the user is brought back to the screen instructing replacement of the sensor (S1514). If the sensor has been replaced and is ready, and the infusion device determines that it is again receiving the sensor signal from the sensor, the infusion device 34 may inform the user that the new sensor is recognized (S1518), and subsequently display the MAIN MENU screen (S1502). Otherwise, if the new sensor is not successfully recognized by the infusion device, the infusion device 34 once again informs the user that the sensor is still not detected (S1512).

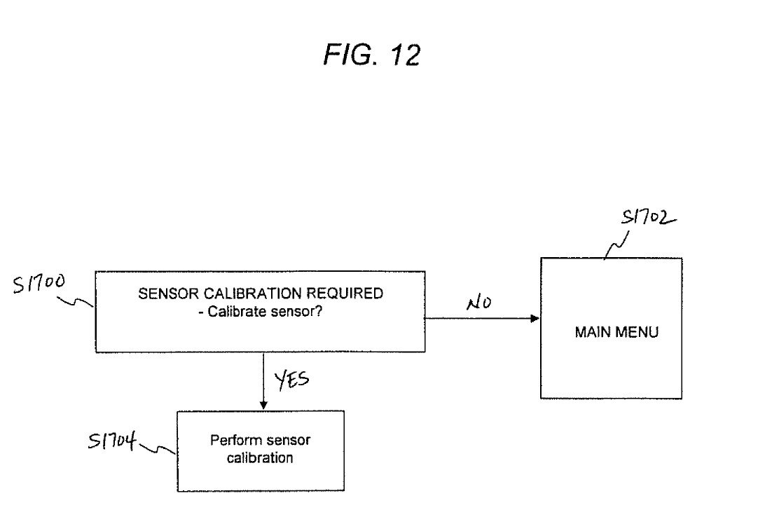

FIG. 11 illustrates a method for guiding user action when a weak sensor signal is detected in accordance with one embodiment of the present invention. Similar to the LOST SENSOR alarm described above, if the infusion device 34 fails to receive the sensor signal from the transmitter 30 for a certain period of time that is shorter than the predetermined period of time that triggers the LOST SENSOR alarm (e.g., 15, 20, or 30 minutes), then the infusion device 34 may display a WEAK SIGNAL alarm screen to notify the user that the infusion device 34 has not received sensor data from the transmitter 30 for that period of time (S1600). The period of time associated with the WEAK SIGNAL alarm may be a default value or a value set by the user. Concurrently with or after the WEAK SIGNAL alarm screen is displayed, the user is asked whether the cause of the weak sensor signal is to be found (S1600). If the answer is “no,” a MAIN MENU screen is displayed (S1602). If the answer is “yes,” the infusion device 34 ascertains the cause of the weak signal. For example, the cause of the weak signal may be that the sensor is too far away from the infusion device. As such, the user is informed of this information and is instructed to move the infusion device closer to the sensor (S1604). If the signal is strengthened by moving the infusion device closer to the sensor and the infusion device determines that it is again receiving the sensor signal from the sensor, the infusion device 34 may inform the user of the strengthened signal (S1608), and then display the MAIN MENU screen (S1602).

Alternatively, the infusion device 34 may ask the user if the infusion device has been moved (S1606). If the answer is “no,” the user is brought back to the screen instructing the infusion device to be moved (S1604). If the answer is “yes,” the infusion device may determine whether it is again receiving the sensor signal from the sensor, and if so, the infusion device 34 informs the user of the strengthened signal (S1608), and subsequently displays the MAIN MENU screen (S1602). Otherwise, if the signal is not strengthened, the infusion device 34 once again asks to find the cause of the weak sensor signal (S1600).

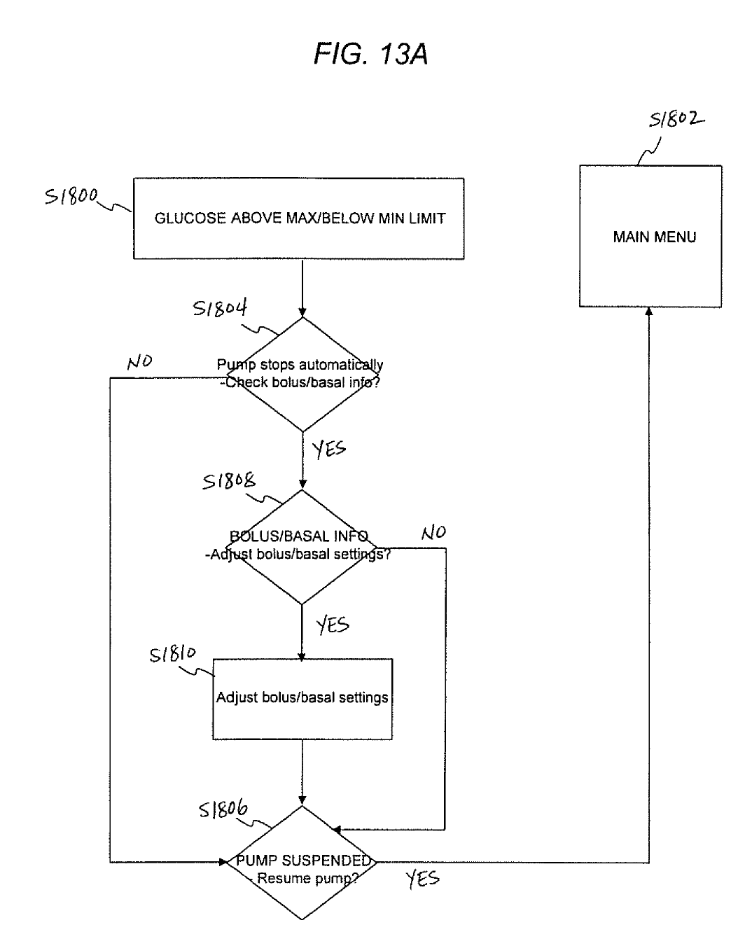

FIG. 12 illustrates a method for guiding user action when sensor calibration is required in accordance with one embodiment of the present invention. As described above, the sensor 26 generates a sensor signal representative of blood glucose levels in the user's body, and provides the sensor signal to the telemetered characteristic monitor transmitter 30, which in turn transmits the sensor signal to the infusion device 34. However, the sensor 26 periodically requires a blood glucose reference value to calibrate the sensor 26. Thus, the user performs a finger stick using a blood glucose meter, and then enters the blood glucose meter value to calibrate the sensor. Referring to FIG. 12, a SENSOR CALIBRATION REQUIRED alarm screen is displayed when the infusion device 34 requires a sensor to be calibrated (S1700). Accordingly, the user is asked whether the user would like to calibrate the sensor (S1700). If the answer is “no,” a MAIN MENU screen is displayed (S1702). If the answer is “yes,” the user is then instructed to perform a finger stick using a blood glucose meter, and guided through one or more screens to enter the blood glucose meter value into the infusion device in order to calibrate the sensor (S1704).

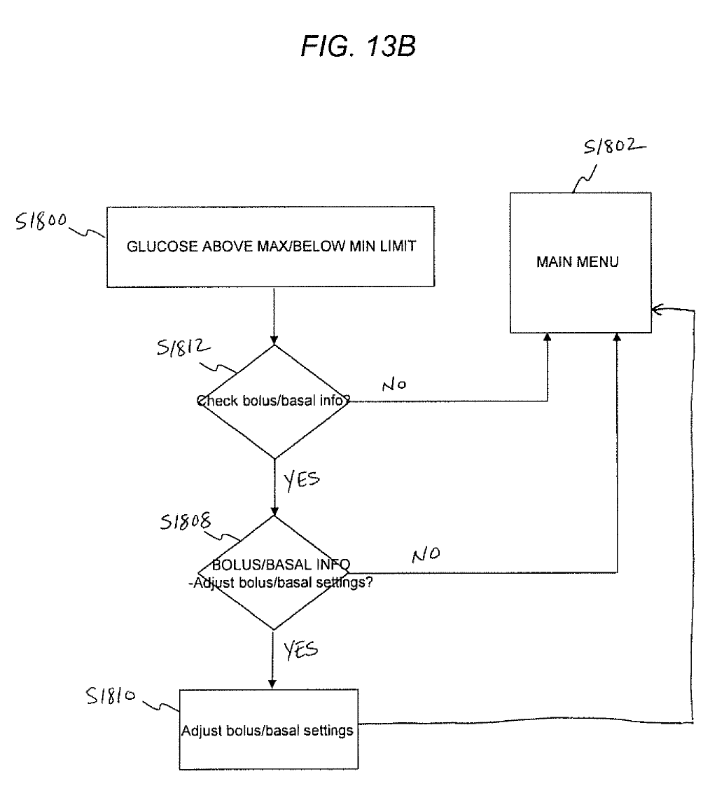

FIGS. 13A and 13B illustrate methods for guiding user action when a user glucose level above a maximum or below a minimum boundary is detected in accordance with one embodiment of the present invention. Referring to FIG. 13A, a GLUCOSE ABOVE MAXIMUM/BELOW MINIMUM LIMIT alarm screen is displayed when the infusion device 34 detects a glucose level that is beyond a predefined maximum or minimum boundary (S1800). Here the maximum/minimum boundary may be a default value or a value set by the user.

In accordance with one embodiment of the present invention, the infusion device 34 automatically stops pumping insulin to the body when the detected glucose level is below the predefined boundary. As such, the infusion device 34 informs the user of this information and asks if the user desires to examine any information or settings on the infusion device related to the user's therapy management, such as bolus-related or basal-related information (S1804). If the user answers “no,” a PUMP SUSPENDED screen is displayed, wherein the user is asked to resume pumping of insulin (S1806). If the user answers “yes” to the PUMP SUSPENDED screen, pumping is resumed and a MAIN MENU screen is subsequently displayed (S1802).

Alternatively, if the user wishes to examine the bolus-related/basal-related information, a BOLUS/BASAL INFORMATION screen is displayed, wherein the user is asked to adjust any bolus-related or basal-related settings (S1808). If the user answers “no,” the PUMP SUSPENDED screen is displayed (S1806). However, if the user answers “yes,” the user is instructed to adjust any desired bolus-related or basal-related settings (S1810). For example, the user may be presented with the options of setting a temporary basal rate that is lower than the currently programmed basal rate, modifying the currently programmed basal rate pattern, or programming a meal bolus as the user will be ingesting carbohydrates to correct his/her low blood glucose level. Once the adjustments are completed, the PUMP SUSPENDED screen is displayed, wherein the user is asked to resume pumping insulin according to the adjusted settings (S1806). If the user answers “yes,” pumping is resumed and the MAIN MENU screen is displayed (S1802).

In other alternative embodiments, instead of automatically stopping the delivery of insulin to the body when the detected glucose level is below the predefined boundary in S1804, the infusion device may ask the user whether to stop insulin delivery. If the user answers “yes,” the infusion device may stop the delivery of insulin to the body and then continue with the logic illustrated in FIG. 13A from S1804 and on. But if the user answers “no,” the infusion device may continue the delivery of insulin according to the existing settings, and then display the MAIN MENU screen (S1802).

Referring to FIG. 13B, in accordance with another embodiment of the present invention, the infusion device 34 continues to pump insulin to the body when the detected insulin level is above the predefined boundary. As such, the infusion device asks if the user desires to examine any information or settings on the infusion device related to the user's therapy management information, such as bolus-related or basal-related information (S1812). If the user answers “no,” the MAIN MENU screen is displayed (S1802). If the user answers “yes,” a BOLUS/BASAL INFORMATION screen is displayed, wherein the user is asked to adjust any bolus-related or basal-related settings (S1808). If the user answers “no,” the MAIN MENU screen is immediately displayed (S1802). But if the user answers “yes,” the user is instructed to adjust any desired bolus-related or basal-related settings (S1810). For example, the user may be presented with the options of setting a temporary basal rate that is higher than the currently programmed basal rate, modifying the currently programmed basal rate pattern, or programming a correction bolus to correct his/her high blood glucose level. Once the adjustments are completed, the MAIN MENU screen is displayed, and the infusion device continues to deliver insulin according to the adjusted settings (S1802).

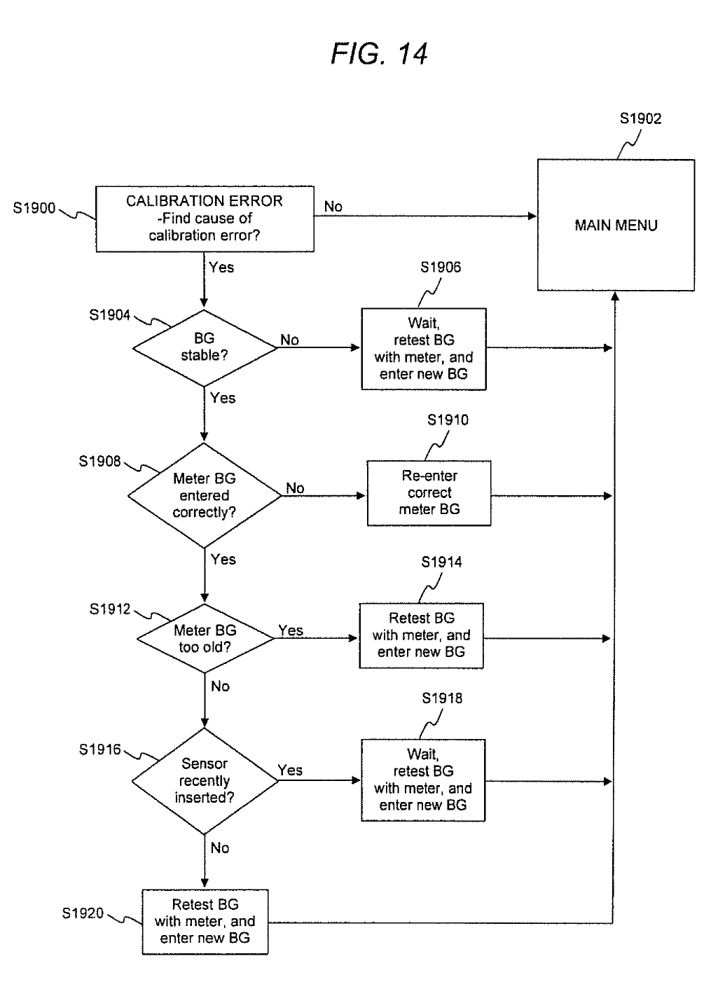

FIG. 14 illustrates a method for guiding user action when a sensor calibration error occurs in accordance with one embodiment of the present invention. Referring to FIG. 14, a CALIBRATION ERROR alarm screen is displayed when the infusion device 34 detects that a calibration error has occurred after the user has entered a blood glucose meter value for calibrating the sensor (S1900). Accordingly, the user is asked whether the user would like to find the cause of the calibration error (S1900). If the answer is “no,” a MAIN MENU screen is displayed (S1902). If the answer is “yes,” the user is asked if his/her blood glucose level is currently stable. The glucose level in the interstitial fluid tends to lag behind the blood plasma glucose level. As a result, the optimum time to calibrate the sensor is when the user's blood glucose level is stable. However, the user may attempt to calibrate the sensor when his/her blood glucose level is unstable, such as following a meal, delivery of an insulin dosage, exercise, or the like. Thus, if the user enters a blood glucose meter value when his/her blood glucose level is unstable, calibration errors are more likely to occur. Accordingly, if the user answers “no,” indicating that his/her blood glucose level is currently unstable, the user is instructed to wait for a short period of time (for example, 1 to 2 hours) and then perform another finger stick using a blood glucose meter and enter the new blood glucose meter value into the infusion device to calibrate the sensor (S1906). A MAIN MENU screen is displayed shortly thereafter (S1902). In particular embodiments, the infusion device may remind the user to perform another finger stick after the recommended waiting period has elapsed, and then guide the user through one or more screens to enter the new blood glucose meter value into the infusion device to calibrate the sensor.

However, if the user answers “yes,” indicating that his/her blood glucose level is stable, the user is asked whether the blood glucose meter value was correctly entered into the infusion device (S1908). If the user answers “no,” the user is instructed to reenter the correct blood glucose meter value into the infusion device (S1910) so that the sensor can be calibrated with the correct blood glucose meter value, and a MAIN MENU screen is subsequently displayed (S1902).

Alternatively, if the user answers “yes”, the user is asked whether the blood glucose meter value entered into the infusion device was too old to be used for calibration (S1912). For example, in one embodiment, the blood glucose meter value may be used for calibrating the sensor only if it is less than 10 to 15 minutes old. If the blood glucose meter value is older than that, it may not accurately reflect the user's current blood glucose level, and as a result, cause a calibration error. If the user answers “yes,” indicating that the blood glucose meter value entered into the infusion device was too old, the user is instructed to perform another finger stick using a blood glucose meter, and guided through one or more screens to enter the new blood glucose meter value into the infusion device to calibrate the sensor (S1914). A MAIN MENU screen is displayed shortly thereafter (S1902).

However, if the user answers “no,” indicating that the blood glucose meter value entered into the infusion device was not too old, the user is then asked whether the sensor was recently inserted (S1916). If a calibration error occurs shortly after a sensor has been recently inserted, it indicates that the sensor needs additional time to stabilize. Accordingly, if the user answers “yes,” indicating that the sensor was recently inserted, the user is instructed to wait for a short period of time (for example, 15, 30, or 45 minutes to 1 or 2 hours) and then perform another finger stick using a blood glucose meter and enter the new blood glucose meter value into the infusion device to calibrate the sensor (S1918). A MAIN MENU screen is displayed shortly thereafter (S1902). In particular embodiments, the infusion device may remind the user to perform another finger stick using a blood glucose meter after the recommended waiting period has elapsed, and then guide the user through one or more screens to enter the new blood glucose meter value into the infusion device to calibrate the sensor.

Alternatively, if the user answers “no,” indicating that the sensor was not recently inserted, then the infusion device instructs the user to perform another finger stick using a blood glucose meter, and then guides the user through one or more screens to enter the new blood glucose meter value into the infusion device to calibrate the sensor (S1920). A MAIN MENU screen is subsequently displayed (S1902).

While the description above refers to particular embodiments of the present invention, it will be understood that many modifications may be made without departing from the spirit thereof. Thus, the accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. In an infusion system including an external infusion device, a sensor, and a transmitter for transmitting a sensor signal from the sensor to the external infusion device, a method for guiding action by a user when the external infusion device fails to receive the sensor signal from the sensor, the method comprising:
displaying an alarm indicating that the infusion device has failed to receive the sensor signal from the sensor for a period of time;
determining a cause of the failure to receive the sensor signal from the sensor; and
directing the user to perform at least one corrective action associated with the failure to receive the sensor signal from the sensor by providing the user with one or more outputs to which the user is to respond with a respective user input or action, wherein each successive one of the outputs is responsive to a previous user input or action.

2. The method of claim 1, wherein the alarm indicates that the infusion device has failed to receive the sensor signal from the sensor for a predetermined period of time.

3. The method of claim 1, wherein the alarm indicates that the infusion device has failed to receive the sensor signal from the sensor for a period of time set by the user.

4. The method of claim 1, wherein the cause of the failure to receive the sensor signal from the sensor relates to the sensor being located too far away from the infusion device, and the directing the user to perform at least one corrective action comprises directing the user to move the infusion device closer to the sensors the method further comprising:
determining whether the infusion device is receiving the sensor signal from the sensor; and
informing the user that the failure to receive the sensor signal from the sensor has been corrected if the infusion device is determined to be receiving the sensor signal from the sensor.

5. The method of claim 4, wherein if the infusion device is determined not to be receiving the sensor signal from the sensor, the directing the user to perform at least one corrective action further comprises directing the user to check a connection between the sensor and the transmitter.

6. The method of claim 5, wherein the directing the user to perform at least one corrective action further comprises directing the user to replace the sensor if the failure to receive the sensor signal from the sensor has not been corrected after directing the user to check the connection between the sensor and the transmitter.

7. In an infusion system including an external infusion device and a sensor, a method for guiding action by a user when the sensor requires the user to perform a calibration, the method comprising:
displaying a notification when the sensor requires a calibration to be performed by the user; and
directing the user to perform the calibration by providing the user with one or more outputs to which the user is to respond with a respective user input or action, wherein each successive one of the outputs is responsive to a previous user input or action.

8. The method of claim 7, wherein the directing the user to perform the calibration further comprises instructing the user to obtain a calibration reference value for the sensor and guiding the user to enter the calibration reference value into the infusion device to calibrate the sensor.

9. In an infusion system including an external infusion device for delivering medication to a user and a sensor for measuring glucose levels in the user, a method for guiding action by the user when a glucose level is above a maximum or below a minimum boundary, the method comprising:
displaying an alarm when a glucose level of the user is above a maximum or below a minimum boundary; and
directing the user to perform at least one corrective action associated with the alarm by providing the user with one or more outputs to which the user is to respond with a respective user input or action, wherein each successive one of the outputs is responsive to a previous user input or action.

10. The method of claim 9, wherein the directing the user to perform at least one corrective action further comprises:
asking the user to adjust settings on the infusion device related to therapy management of the user; and
directing the user to adjust one or more of the settings on the infusion device related to the therapy management of the user.

11. The method of claim 10, wherein the settings on the infusion device related to the therapy management of the user comprise at least one of bolus-related settings and basal-related settings.

12. The method of claim 11, wherein the directing the user to adjust one or more of the settings on the infusion device related to the therapy management of the user comprises at least one of programming a temporary basal rate, modifying a basal rate pattern, programming a meal bolus, and programming a correction bolus.

13. The method of claim 10, further comprising:
suspending delivery of the medication to the user when the glucose level of the user is below the minimum boundary; and
resuming delivery of the medication to the user after directing the user to adjust one or more of the settings on the infusion device related to the therapy management of the user.

14. The method of claim 13, wherein the delivery of the medication to the user is suspended automatically when the glucose level of the user is below the minimum boundary.

15. The method of claim 13, wherein the delivery of the medication to the user is suspended based on input from the user.

16. In an infusion system including an external infusion device and a sensor, a method for guiding action by a user when a calibration reference value for the sensor entered by the user triggers a calibration error, the method comprising:
displaying an alarm when the calibration reference value for the sensor entered by the user triggers the calibration error;
determining a cause of the calibration error; and
directing the user to perform at least one corrective action associated with the calibration error by providing the user with one or more outputs to which the user is to respond with a respective user input or action, wherein each successive one of the outputs is responsive to a previous user input or action.

17. The method of claim 16, wherein the determining the cause of the calibration error comprises asking whether glucose levels of the user are stable, and if the glucose levels of the user are not stable, the directing the user to perform at least one corrective action further comprises instructing the user to wait for a period of time, instructing the user to obtain a calibration reference value, and guiding the user to enter the calibration reference value into the infusion device to calibrate the sensor.

18. The method of claim 16, wherein the determining the cause of the calibration error comprises asking whether the calibration reference value for the sensor was correctly entered by the user, and if the calibration reference value for the sensor was not correctly entered, the directing the user to perform at least one corrective action further comprises instructing the user to reenter the calibration reference value into the infusion device to calibrate the sensor.

19. The method of claim 16, wherein the determining the cause of the calibration error comprises asking whether the calibration reference value for the sensor was too old to calibrate the sensor, and if the calibration reference value for the sensor was too old to calibrate the sensor, the directing the user to perform at least one corrective action further comprises instructing the user to obtain a new calibration reference value for the sensor and guiding the user to enter the new calibration reference value into the infusion device to calibrate the sensor.

20. The method of claim 16, wherein the determining the cause of the calibration error comprises asking whether sensor was recently inserted into the user, and if the sensor was recently inserted into the user, the directing the user to perform at least one corrective action further comprises instructing the user to wait for a period of time, instructing the user to obtain a new calibration reference value for the sensor, and guiding the user to enter the new calibration reference value into the infusion device to calibrate the sensor.
